(19) United States
(12) Patent Application Publication
(10) Pub. No.: US 2004/0229867 A1
(43) Pub. Date: Nov. 18, 2004

(54) Treatment of gastrointestinal dysfunction and related stress with an enantiomerically-pure (R) 2,3-benzodiazepine

(76) Inventor: Robert F. Kucharik, Glenmoore, PA (US)

Correspondence Address: BELL & ASSOCIATES, 416 FUNSTON ST., SUITE 100, SAN FRANCISCO, CA, 94118, US

(21) Appl. No.: 10/846,822

(22) Filed: May 13, 2004

Related U.S. Application Data
(60) Provisional application No. 60/471,160, filed on May 16, 2003.

(51) Int. Cl. A61K 31/5513

(52) U.S. Cl. 514/221

(57) ABSTRACT

The present invention relates to methods of treatment for symptoms of gastrointestinal dysfunction and related stress that are frequently associated with, for example, irritable bowel syndrome. The symptoms include altered bowel motility, gastrointestinal inflammation, visceral hypersensitivity, or gastric ulcers.

The present application claims the benefit of U.S. Provisional Patent Application Serial No. 60/471,160 entitled Treatment Of Gastrointestinal Dysfunction And Related Stress With Enantiomerically-Pure 2,3-Benzodiazepines, filed May 16, 2003, which is herein incorporated by reference in its entirety for all purposes.

### FIELD OF THE INVENTION

The present invention relates to methods of treatment for symptoms of gastrointestinal dysfunction and related stress that are frequently associated with, for example, irritable bowel syndrome.

### BACKGROUND OF THE INVENTION

Tofisopam is a racemic mixture of (R)— and (S)-enantiomers. This is due to the asymmetric carbon, i.e., a carbon with four different groups attached, at the 5-position of the benzodiazepine ring. Tofisopam is a non-sedative anxiolytic that has no appreciable sedative, muscle relaxant or anticonvulsant properties (Horvath et al., Progress in Neurobiology, 60(4): 309-342 (2000)). In addition, tofisopam has been used in the treatment of gastrointestinal disorders, including irritable bowel syndrome.

The molecular structure and conformational properties of tofisopam have been determined by nuclear magnetic resonance (NMR), circular dichroism (CD), and x-ray crystallography (Visy et al., Chirality 1: 271-275 (1989)). The 2,3-diazepine ring exists as two different conformers. The major tofisopam conformers, (+)R and (−)S, contain a 5-ethyl group in a quasi-equatorial position. The 5-ethyl group is positioned quasi-axially in the minor conformers, (−)R and (+)S. Thus, racemic tofisopam can exist as four molecular species, i.e., two enantiomers, each of which exists as two conformations. The sign of the optical rotation is reversed upon inversion of the diazepine ring from one conformer to the other. In crystal form, tofisopam exists only as the major conformations, with dextrorotatory tofisopam being of the (R) absolute configuration. (Toth et al., J. Heterocyclic Chem., 20: 709-713 (1983); Fogassy et al., Bioorganic Heterocycles, Van der Plas, H. C., Ötvös, L, Simongi, M., editors, Budapest Amsterdam: Akademia; Kiado-Elsevier, 229: 233 (1984)).

Differential binding of the (+) and (−) conformations of tofisopam has been reported in binding studies with human albumin (Simongi et al. Biochem. Pharm., 32(12): 1917-1920 (1983)). The two (±) conformers have also been reported as existing in equilibrium (Zsila et al., Journal of Liquid Chromatography & Related Technologies, 22(5); 713-719, 1999; and references therein).

The (R)- enantiomer of tofisopam (R)-1-(3,4-dimethoxyphenyl)-4-methyl-5-ethyl-7,8-dimethoxy-5H-2,3-benzodiazepine) has been isolated and shown to possess the nonsedative anxiolytic activity of the racemic mixture. See U.S. Pat. No. 6,080,736; the entire disclosure of which is incorporated herein by reference.

Irritable bowel syndrome (IBS) is a common disorder that has a pronounced effect on the quality of life and that accounts for a large proportion of healthcare costs. IBS is defined on the basis of the recently modified Rome criteria as (A) the presence for at least 12 weeks (not necessarily consecutive) in the preceding 12 months of abdominal discomfort or pain that cannot be explained by structural or biochemical abnormalities, and (B) at least two of the following three (1) pain relieved with defecation; (2) pain, when the onset thereof is associated with a change in the frequency of bowel movements (diarrhea or constipation); and pain, when the onset thereof is associated with a change in the form of the stool (lose, watery, or pellet-like). IBS may be divided into four subcategories according to whether the predominant symptom is abdominal pain, diarrhea, constipation, or constipation alternating with diarrhea.

Approximately 15 percent of U.S. adults report symptoms that are consistent with the diagnosis of the IBS; the disease affects three times as many women as men. Whether this difference reflects a true predominance of the disorder among women or merely the fact women are more likely to seek medical care has not been determined. IBS is the most common diagnosis made by gastroenterologists in the United States and accounts for 12 percent of visits to primary care providers. It is estimated that only 25 percent of persons with this condition seek medical care for it, and studies suggest that those who seek care are more likely to have behavioral and psychiatric problems than are those who do not seek care. In addition, patients with a diagnosis of IBS are at increased risk for other, non-gastrointestinal functional disorders such as fibromyalgia and interstitial cystitis. The irritable bowel syndrome accounts for an estimate $8 billion in direct medical costs and $25 billion in indirect costs annually in the United States.

Converging evidence supports the concept that IBS results from altered regulation of gastrointestinal motility and epithelial function, as well as altered perception of visceral events. See Mayer et al., Digestive Diseases, 19: 212-218 (2001), the entire disclosure of which is incorporated herein by reference.

Altered bowel motility, visceral hypersensitivity, psychosocial factors, an imbalance in neurotransmitters, and infection have all been proposed as playing a part in the development of irritable bowel syndrome. See B. Horwitz et al., The New England Journal of Medicine, 344: 24 (2001), the entire disclosure of which is incorporated herein by reference. Furthermore, gastrointestinal inflammation may be associated with irritable bowel syndrome, along with stress.

New agents are needed which are useful in the treatment of symptoms such as altered bowel motility, visceral hypersensitivity or gastrointestinal inflammation and related stress, associated for example with irritable bowel syndrome. In particular, agents are needed that are appropriate for the treatment and prevention of these symptoms.

### DEFINITIONS

The term “enantiomerically-pure” when used to refer to a compound, means the (R)— or (S)-enantiomers of the compound have been separated such that the composition is 80% or more by weight a single enantiomer. Thus, by “enantiomerically pure (R)-tofisopam” is meant tofisopam that comprises 80% or more by weight of the (R)-enantiomer and likewise contains 20% or less of the (S)-enantiomer as a contaminant, by weight.

The term “effective amount” when used to describe therapy to a patient to treat gastrointestinal dysfunction and related stress, refers to the amount of (S)-tofisopam or (R)-tofisopam that results in a therapeutically useful reduction in the gastrointestinal dysfunction when administered to a patient suffering from a disorder which manifests gastrointestinal dysfunction. The term “individual” or “subject”, includes humans and non-human animals.

### SUMMARY OF THE INVENTION

According to the present invention, enantiomerically-pure (R)-tofisopam and pharmaceutically acceptable salts thereof are useful in methods for treating gastrointestinal dysfunction and related stress.

According to one embodiment of the invention, there is provided a method of treating or preventing altered bowel motility in an individual in need of such treatment, comprising administering to the individual an effective amount of enantiomerically-pure (R)-tofisopam; or a pharmaceutically-acceptable salt of such a compound. Such altered bowel motility may be related to, but is not limited to, irritable bowel syndrome.

According to a third embodiment of the invention, there is provided a method of treating or preventing visceral hypersensitivity, pain and bloating in an individual in need of such treatment, comprising administering to the individual an effective amount of enantiomerically-pure (R)-tofisopam; or a pharmaceutically-acceptable salt of such a compound. Such symptoms may be related to, but is not limited to, irritable bowel syndrome.

In yet a further embodiment, there is provided a method of treating or preventing ulcer formation in an individual in need of such treatment, comprising administering to the individual an effective amount of enantiomerically-pure (R)-tofisopam; or a pharmaceutically-acceptable salt of such a compound. Such ulcer formation may be related to, but is not limited to, irritable bowel syndrome.

### DETAILED DESCRIPTION OF THE INVENTION

According to the present invention, enantiomerically-pure (R)-tofisopam and pharmaceutically acceptable salts thereof is useful in methods for treating gastrointestinal dysfunction and related stress. Surprisingly, it has been shown that the pure enantiomer shows greater effectiveness in a variety of tests of animal models for gastrointestinal dysfunction and related stress compared with the racemic mixture (RS-tofisopam).

For example, (R)-tofisopam has demonstrated therapeutic effectiveness in the Glass Bead Test in mice, an animal model designed to evaluate the ability of compounds to affect stretch-stimulated colonic propulsion. In the test, usually performed in mice, a 3-mm glass bead is inserted through the anus into the distal colon (using a glass rod) to a depth of 2 cm. The time to expel the glass bead is then measured; normally, the glass bead is expelled in approximately 10 minutes. This model is especially sensitive to compounds with inhibitory effects on stretch-stimulated propulsive motor activity; as such, it is often used as an animal model for IBS. A test compound that inhibits stretch-stimulated-colonic propulsive motility, may be used to treat gastrointestinal dysfunction, including IBS. (R)-tofisopam inhibited propulsive motility to a greater extent than the racemate.

Thus, according to one embodiment of the invention, there is provided a method of treating or preventing altered bowel motility in an individual in need of such treatment, comprising administering to the individual an effective amount of at least one compound selected from enantiomerically-pure R-tofisopam; or a pharmaceutically-acceptable salt of such a compound. Such altered bowel motility may be related to, but is not limited to, irritable bowel syndrome.

Another gastrointestinal disorder model tested was the dextran sulfate sodium-induced colitis. In this model of colitis, an acute inflammation of the colon is produced by administration of dextran sulfate sodium (DSS) as a 5% solution in tap water. This colitis is characterized by histological events and an influx of neutrophils, macrophages and mediators of inflammation similar to those observed with human inflammatory bowel diseases. Drugs known to be of useful for treating irritable bowel disease (IBD), such as sulfasalazine, have been shown to have activity in this model. As demonstrated in the Examples provided below, (R)-tofisopam showed a substantially greater effect in this model compared with racemic tofisopam (RS-tofisopam).

According to a second embodiment of the invention, there is provided a method of treating or preventing gastrointestinal inflammation in an individual in need of such treatment, comprising administering to the individual an effective amount of enantiomerically pure R-tofisopam; or a pharmaceutically-acceptable salt of such a compound. Such gastrointestinal inflammation may be related to, but is not limited to, irritable bowel syndrome.

In yet another gastrointestinal disorder model, the visceral pain and bloating test, the ability of (R)-tofisopam to inhibit abdominal contractions in the rat was investigated. (R)-tofisopam was able to inhibit abdominal contractions to a greater extent than racemic tofisopam.

According to a third embodiment of the invention, there is provided a method of treating or preventing visceral hypersensitivity, pain, and bloating in an individual in need of such treatment, comprising administering to the individual an effective amount of enantiomerically pure (R)-tofisopam; or a pharmaceutically-acceptable salt of such a compound. Such visceral hypersensitivity, pain, and bloating may be related to, but is not limited to, irritable bowel syndrome.

In a further model, the use of (R)-tofisopam for reducing ulcer formation was investigated in the water immersion stress test. Stress has been shown to rapidly induce ulcer formation in rats. The water-immersion stress test evaluates the ability of compounds to affect ulcer formation induced by water-immersion stress in rats (West J. Pharmacol. Methods, 8: 33-37 (1982)). Yamaguchi et al. demonstrated significant activity with RS-tofisopam in reducing ulcer formation, noting that pre-treatment with RS-tofisopam (30 or 100 mg/kg PO) reduced the number of stress-induced ulcers and the total area of ulceration by as much as 90% (Yamaguchi et al. Can. J. Physiol. Pharmacol., 61: 619-625 (1983)). Other investigators have examined the effects of RS-tofisopam on various aspects of gastric function in rats. Sato et al. demonstrated that intracerebroventricular injection of RS-tofisopam (50 or 100 μg) increased both basal gastric acid output and mucosal blood flow while intravenous injection of RS-tofisopam (10 mg/kg) did not change basal gastric acid output (Sato et al. Nippon Yakurigaku Zasshi, 79: 307-315 (1982)). Matsuo and Seki showed that RS-tofisopam suppressed the development of hydrochloric acid-induced ulcers and alkali-induced ulcers and promoted the healing of cauterization-induced ulcers (Matsuo and Seki, Yakurito Chiryo 16(8): 157-164 (1988)). (R)-tofisopam inhibited ulcer formation to a greater extent than racemic tofisopam as illustrated in the Examples.

In yet a further embodiment, there is provided a method of treating or preventing gastric ulcer formation in an individual in need of such treatment, comprising administering to the individual an effective amount of enantiomerically pure (R)-tofisopam; or a pharmaceutically-acceptable salt of such a compound. Such gastric ulcer formation may be related to, but is not limited to, irritable bowel syndrome.

Furthermore, a multiplicity of gastrointestinal dysfunction symptoms may be treated with either (R)-tofisopam at the same time. For example, (R)-tofisopam may be employed for treating the symptoms of altered bowel motility and gastrointestinal inflammation. As another example, (R)-tofisopam may be used for treating gastrointestinal stress and visceral pain. In another example, (R)-tofisopam is employed for the treatment of irritable bowel syndrome. Neither of these examples should be taken to limit other treatment possibilities provided by this invention.

(R)-tofisopam useful in the present invention may be prepared by one of several methods. These methods generally begin with synthetic strategies and procedures used in the synthesis of racemic tofisopam and further include a resolution of racemic tofisopam to isolate the (R)-enantiomer. See U.S. Pat. Nos. 3,736,315 and 4,423,044 (tofisopam syntheses) and Horvath et al., Progress in Neurobiology, 60(4): 309-342 (2000) and references cited therein (preparation of tofisopam and analogs thereof), the entire disclosures of which are incorporated herein by reference. In the synthesis methods that follow, the product of the chemical syntheses is racemic tofisopam. This racemic mixture is subsequently separated using known methods of resolution to produce the enantiomerically pure (R)-tofisopam. Preferably, the compound used in methods of the present invention has a composition that is 85% by weight or greater of the desired enantiomer, and 15% by weight, or less, of the undesired enantiomer. More preferably, the compound used in methods of the present invention has a composition that is 90% by weight or greater of the desired enantiomer and 10% by weight, or less, of the undesired enantiomer. More preferably, the compound used in methods of the present invention has a composition that is 95% by weight or greater of the desired enantiomer and 5% by weight, or less, of the corresponding undesired enantiomer. Most preferably, the compound used in methods of the present invention has a composition that is 99% by weight or greater of the desired enantiomer and 1% by weight, or less, of the corresponding undesired enantiomer.

The synthetic procedures shown (or referenced) above produce racemic tofisopam. In order to prepare (R)-tofisopam useful in methods of the present invention, the racemic mixture must be resolved.

Racemic tofisopam may, for example, be converted to the (S)-dibenzoyltartaric acid salt, which product is a diastereomeric mixture of SS and RS configurations. The pair of diastereomers (R,S) and (S,S) possess different properties, for example, differential solubilities, that allow for the use of conventional separation methods. Fractional crystallization of diastereomeric salts from a suitable solvent is one such separation method. This resolution has been successfully applied to the resolution of racemic tofisopam. See Hungarian Patent 178516 and also Toth et al., J. Heterocyclic Chem., 20: 709-713 (1983), the entire disclosures of which are incorporated herein by reference.

Racemic tofisopam may also be resolved without diastereomer formation by differential absorption on a chiral stationary phase of a chromatography column, particularly a preparative HPLC column. Chiral HPLC columns are commercially available with a variety of packing materials to suit a broad range of separation applications. Exemplary stationary phases suitable for resolving the racemic 2,3-benzodiazepines include:

(i) macrocyclic glycopeptides, such as silica-bonded vancomycin which contains 18 chiral centers surrounding three pockets or cavities;

(ii) chiral α₁-acid glycoprotein;

(iii) human serum albumin; and

(iv) cellobiohydrolase (CBH).

Chiral α₁-acid glycoprotein is a highly stable protein immobilized onto spherical silica particles that tolerates high concentrations of organic solvents, high and low pH, and high temperatures. Human serum albumin, though especially suited for the resolution of weak and strong acids, zwitterionic and nonprotolytic compounds, has been used to resolve basic compounds. CBH is a very stable enzyme that has been immobilized onto spherical silica particles and is preferentially used for the separation of enantiomers of basic drugs from many compound classes.

The resolution of tofisopam by chiral chromatography using macrocyclic glycopeptide as a stationary phase on a Chirobiotic V™ column (ASTEC, Whippany, N.J.) is disclosed in U.S. Pat. No. 6,080,736. Fitos et al. (J. Chromatogr., 709: 265 (1995)), discloses another method for resolving racemic tofisopam by chiral chromatography using a chiral α₁-acid glycoprotein as a stationary phase on a CHIRAL-AGP™ column (ChromTech, Congleton, Cheshire, UK). This method separates the (R)— and (S)-enantiomers and also resolves the two conformers (discussed below) of each enantiomer. The Chirobiotic V™ column is available in a semi-preparative size as employed for the above separation 500 mm×10 mm). In addition, the stationary phase of the Chirobiotic V™ column is commercially available in bulk for packing of preparative chromatography columns with larger sample capacity. The entire disclosures of the aforementioned patents and publications are incorporated herein by reference in their entireties.

In addition to existing as (R)— and (S)-enantiomers, tofisopam also exists in two stable conformations that may be assumed by the benzodiazepine ring as generally depicted below.

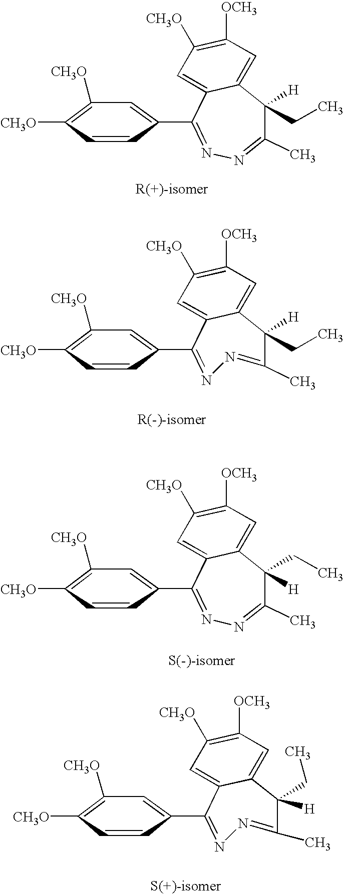

The present invention includes methods as described herein that use any and all observable conformations of (R)-tofisopam.

Yet further, the present invention includes methods that use a prodrug of (R)-tofisopam. Prodrugs according to this invention are inactive derivatives of R-tofisopam that are metabolized in vivo into the active agent in the body. Prodrugs useful according to this invention are those that have substantially the same or better therapeutic value than R-tofisopam in treating or preventing convulsions or seizures. For example, a prodrug useful according to this invention can improve the penetration of the drug across biological membranes leading to improved drug absorption; prolong duration of the action of the drug, for example, slow release of the parent drug from the prodrug and/or decrease first-pass metabolism of the drug; target the drug action; improve aqueous solubility and stability of the drug (for example, intravenous preparations, eyedrops, etc.); improve topical drug delivery (for example, dermal and ocular drug delivery); improve the chemical and/or enzymatic stability of drugs (for example, peptides); or decrease side effects due to the drug. Methods for making prodrugs are know in the art (for example, Balant, L. P., Eur. J. Drug Metab. Pharmacokinet. 15: 143-153 (1990); and Bundgaard, H., Drugs of the Future 16: 443-458 (1991); incorporated by reference herein).

(R)-tofisopam may similarly be used in the methods of this invention in combination with a second drug. The second drug may include another drug effective in treating IBS, such as 5HT3 antagonists, 5HT4 agonists, antispasmodics, antidiarrheals, laxatives, SSRIs, TCAs, CCK A antagonists, M3 antagonists, opioid mu antagonists, 5HT3 antagonists/5HT4 agonists, neurokinin 2 antagonists, opiod kappa agonists, neurokinin 3 antagonists, neurokinin 1 antagonists, opioid delta agonists, CRF antagonists, NSRIs, chloride channel agonists, chloride channel antagonists, 5HT1a agonists, GLP-1 agonists, CCK B antagonists/gastrin antagonists, beta 3 agonists, calcium channel antagonists, M1 antagonists, D2 agonists/5HT4 agonists, integrin antagonists, and purine nucleoside phosphorylase inhibitors.

(R)-tofisopam used in the practice of methods of the present invention may take the form of pharmaceutically-acceptable salts. The term “salts”, embraces salts commonly used to form alkali metal salts and to form addition salts of free acids or free bases. The term “pharmaceutically-acceptable salt” refers to salts that possess toxicity profiles within a range so as to have utility in pharmaceutical applications. Pharmaceutically unacceptable salts may nonetheless possess properties such as high crystallinity, which have utility in the practice of the present invention, such as for example utility in a synthetic process or in the process of resolving enantiomers from a racemic mixture. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, beta-hydroxybutyric, salicyclic, galactaric, and galacturonic acid.

The compounds useful in methods of the invention may be administered to individuals (mammals, including animals and humans) afflicted with disorders associated with elevated body temperature or with disorders wherein lowering the body temperature below the normal body temperature has therapeutic benefit.

For treating or preventing irritable bowel syndrome, the specific dose of compound according to the invention to obtain therapeutic benefit will, of course, be determined by the particular circumstances of the individual patient including, the size, weight, age and sex of the patient. Also determinative will be the nature and stage of the disease and the route of administration. For example, a daily dosage of from about 100 to 1500 mg/kg/day may be utilized. Preferably, a daily dosage of from about 100 to 1000 mg/kg/day may be utilized. More preferably, a daily dosage of from about 100 to 500 mg/kg/day may be utilized. Higher or lower doses are also contemplated.

For prophylactic administration, (R)-tofisopam should be administered far enough in advance of a known event that increases the body temperature, such that the compound is able to reach the site of action in sufficient concentration to exert a hypothermic effect. The pharmacokinetics of specific formulations may be determined by means known in the art and tissue levels of (R)-tofisopam in a particular individual may be determined by conventional analyses.

The methods of the present invention may comprise administering (R)-tofisopam in the form of a pharmaceutical composition, in combination with a pharmaceutically acceptable carrier. The active ingredient in such formulations may comprise from 0.1 to 99.99 weight percent. By “pharmaceutically acceptable carrier” is meant any carrier, diluent, or excipient that is compatible with the other ingredients of the formulation and not deleterious to the recipient.

(R)-tofisopam may be administered for therapeutic effect by any route, for example enteral (for example, oral, rectal, intranasal, etc.) and parenteral administration. Parenteral administration includes, for example, intravenous, intramuscular, intraarterial, intraperitoneal, intravaginal, intravesical (for example, into the bladder), intradermal, topical, or subcutaneous administration. Also contemplated within the scope of the invention is the instillation of drug in the body of the patient in a controlled formulation, with systemic or local release, such as, for example, in the gastrointestinal tract, of the drug to occur at a later time.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, editor, Remington's Pharmaceutical Sciences, 18th edition, (1990) Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water-soluble salt of the active agent. Stabilizing agents, antioxidizing agents and preservatives may also be added. Suitable antioxidizing agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents, or lubricating agents. According to one tablet embodiment, the active agent may be combined with carboxymethylcellulose calcium, magnesium stearate, mannitol and starch, and then formed into tablets by conventional tableting methods.

The compositions of the present invention can also be formulated so as to provide slow or controlled-release of the active ingredient therein. In general, a controlled-release preparation is a composition capable of releasing the active ingredient at the required rate to maintain constant pharmacological activity for a desirable period of time. Such dosage forms can provide a supply of a drug to the body during a predetermined period of time and thus maintain drug levels in the therapeutic range for longer periods of time than other non-controlled formulations.

For example, U.S. Pat. No.5,674,533 discloses controlled-release compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 discloses a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 discloses controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. The patents cited above are incorporated herein by reference.

Biodegradable microparticles can be used in the controlled-release formulations of this invention. For example, U.S. Pat. No. 5,354,566 discloses a controlled-release powder that contains the active ingredient. U.S. Pat. No. 5,733,566 describes the use of polymeric microparticles that release antiparasitic compositions. These patents are incorporated herein by reference.

The controlled-release of the active ingredient may be stimulated by various inducers, for example, pH, temperature, enzymes, water, or other physiological conditions or compounds. Various mechanisms of drug release exist. For example, in one embodiment, the controlled-release component can swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient (for example, (R)-tofisopam or a pharmaceutically-acceptable salt thereof) in the pharmaceutical composition. In another embodiment, the controlled-release component is biodegradable, induced by exposure to the aqueous environment, pH, temperature, or enzymes in the body. In another embodiment, sol-gels can be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

(R)-tofisopam is administered according to the present invention to patients suffering from conditions that manifest the symptoms of altered bowel mobility, gastrointestinal inflammation, visceral hypersensitivity or gastric ulcers. Such conditions include for example, irritable bowel syndrome or irritable bowel disorder.

The practice of the invention is illustrated by the following non-limiting examples.

### EXAMPLES

### Example 1

### Preparation of (R)-Tofisopam

A. Synthesis of Racemic Tofisopam:

4.41 g (10 mmol) of 1-(3,4-dimethoxyphenyl)-3-methyl-4-ethyl-6,7-dimethoxyisobenzopyrilium chloride hydrochloride is dissolved in methanol (35 mL) at a temperature of 40° C. After cooling to 20-25° C., hydrazine hydrate (0.75 g, 15 mmol, dissolved in 5 mL methanol) is added. The reaction is monitored by HPLC and when complete, is evaporated to dryness. The residue is triturated with cold water (3 mL), filtered and dried to yield the crude (R,S)-1-(3,4-dimethoxyphenyl)-4-methyl-5-ethyl-7-hydroxy-8-methoxy-5H-2,3-benzo-diazepine which is subsequently triturated with hot ethyl acetate to yield the pure product.

B. Resolution of Racemic Tofisopam

The enantiomers of tofisopam were resolved by chiral chromatography. For example, tofisopam (42.8 mg dissolved in acetonitrile (ACN)) was loaded onto a Chirobiotic V™ column (ASTEC, Whippany, N.J.). Elution of the compounds with methyl-tert-butyl ether (MTBE)/ACN 90/10 (v/v), 40 mL/min, was monitored at 310 nm, 2 mm path. The R(+) enantiomer was the first compound to elute from the column. R(−) tofisopam (“peak A′”), S(±) tofisopam (“peak B” and “peak B′”), and residual R(+) tofisopam (“A”) co-eluted and were collected in a subsequent fraction.

The final preparations of R-tofisopam were assayed for enantiomeric purity. R-tofisopam was greater than 97.5% pure (i.e., enantiomeric excess of >95%), and S-tofisopam was 87% pure (i.e., enantiomeric excess of 74%), as determined by analytical chromatography. Analytical evaluations of the starting material and final preparations of R-tofisopam were carried out using Chiral Tech OD GH060 columns (Daicel (USA) Inc., Fort Lee, N.J.) (hexane/IPA 90/10, 25° C., detection at 310 nm).

### Example 2

### Evaluation of Colonic Propulsion Using (R)-Tofisopam

The glass bead test is commonly used to evaluate the ability of compounds to affect stimulated colonic propulsion. In the test, usually performed in mice, a 3-mm glass bead is inserted through the anus into the distal colon (using a glass rod) to a depth of 2 cm. The time to expel the glass bead is then measured; normally, the glass bead is expelled in approximately 10 minutes. This model is especially sensitive to compounds with inhibitory effects on stretch-stimulated propulsive motor activity; as such, it is often used as an animal model for IBS.

In each study, RS— and R-tofisopam were evaluated for their ability to increase or decrease the time for expulsion of the glass bead. Test compounds were administered 30 minutes prior to insertion of the bead, and a maximum time cutoff of 30 minutes to expulsion was used. Data from the experiment are shown in Table 1.

TABLE 1
Effects of Racemic, R-Tofisopam on Colonic Propulsive
Motility in the Glass Bead Test in the Mouse

| | Dose, | Expulsion Time | |
|---|---|---|---|
| Compound | mg/kg IP | Mean ± SEM | % Inhibition |
| Vehicle | 0 | 9.6 ± 2.4 | — |
| RS-tofisopam | 64 | 26.4 ± 2.4** | 82 |
| | 32 | 23.7 ± 2.6 | 71 |
| | 16 | 19.0 ± 2.8 | 46 |
| R-tofisopam | 64 | 28.0 ± 1.1* | 90 |
| | 32 | 29.7 ± 0.3*** | 99 |
| | 16 | 30.0 ± 0.0*** | 100 |

*Significant difference from vehicle,
*p < 0.05,
**p < 0.01, or
***p < 0.001

As seen in the above table, R-tofisopam significantly prolonged (i.e., slowed) colonic expulsion, with R-tofisopam producing near maximal inhibition at most doses tested.

### Example 3

### Charcoal Meal Test Using (R)-Tofisopam

The charcoal meal test is a standard method for evaluating the effect of compounds on basal, nonstimulated propulsive motility of the stomach and small intestine. It is generally considered predictive of a compound's ability to inhibit or accelerate basal gastric propulsion in humans. In the test, usually performed in rats, nonactivated charcoal powder is administered by oral gavage.

Sixty minutes after administration of the charcoal suspension, rats were sacrificed by CO₂ inhalation, the stomach and small intestine were removed, and the distance between the pylorus and the furthest progression of the charcoal meal was measured and compared to the distance between the pylorus and the ileocecal junction. This model is especially sensitive to compounds with the ability to increase or decrease basal propulsive motor activity of the stomach and small intestine. It is therefore often used to predict the potential for compounds to exert antidiarrheal or anticonstipatory effects, as well as to predict the potential for compounds to produce diarrhea or constipation as a side-effect. Two charcoal meal tests were performed in rats. In each study, RS— and R-tofisopam were evaluated for their ability to increase or decrease the percent of the intestine traversed by a charcoal meal. Test compounds were administered 30 minutes prior to administration of the charcoal meal. Data from the first experiment are shown in Table 2.

TABLE 2
Effects of RS- and R-Tofisopam on Gastrointestinal Propulsive
Motility in the Charcoal Meal Test in the Rat

| | Dose, | % Intestine Traversed, | |
|---|---|---|---|
| Treatment | mg/kg IP | Mean ± SEM | % Inhibition^{a} |
| Vehicle | 0 | 81.5 ± 2.8 | — |
| RS-Tofisopam | 64 | 81.7 ± 2.8 | 0 |
| | 32 | 77.7 ± 2.3 | 4.7 |
| | 16 | 73.5 ± 3.5 | 9.8 |
| R-Tofisopam | 64 | 72.9 ± 2.9* | 10.6 |
| | 32 | 85.4 ± 2.9 | (5.0) |
| | 16 | 82.8 ± 3.1 | (1.5) |

*Significant inhibition of gastrointestinal propulsive motility,
p < 0.05
^{a}% inhibition = 100 − (% intestine traversed by treatment ÷ % intestine traversed by vehicle control); numbers in parentheses indicate % increase

As shown in Table 2, none of the compounds had a large or consistent effect on the percentage of the intestine traversed by a charcoal meal. A second experiment was then performed, using a wider dose range and including loperamide as a positive control. Data from this experiment are shown in Table 3.

TABLE 3
Effects of Loperamide, RS-, R-Tofisopam on Gastrointestinal
Propulsive Motility in the Charcoal Meal Test in the Rat

| | Dose, | % Intestine Traversed | |
|---|---|---|---|
| Treatment | mg/kg IP | Mean ± SEM | % Inhibition^{a} |
| Vehicle | 0 | 74.7 ± 3.3 | — |
| Loperamide | 16 | 28.1 ± 2.2* | 62.5 |
| RS-tofisopam | 32 | 65.6 ± 4.5 | 12.2 |
| | 16 | 70.4 ± 3.7 | 5.8 |
| | 8 | 78.3 ± 1.7 | (4.8) |
| | 4 | 77.9 ± 2.9 | (4.2) |
| R-tofisopam | 32 | 59.5 ± 3.2* | 20.3 |
| | 16 | 65.7 ± 3.0 | 12.1 |
| | 8 | 75.2 ± 4.1 | (0.7) |
| | 4 | 71.72 ± 3.2 | 4.1 |

*Significant inhibition of gastrointestinal propulsive motility,
p < 0.05
^{a}% inhibition = 100 − (% intestine traversed by treatment ÷ % intestine traversed by vehicle control); numbers in parentheses indicate % increase

In this experiment, at the highest dose tested, R-tofisopam produced only a slight inhibition of charcoal meal transit. Loperamide, in contrast, produced considerable slowing of the transit of the charcoal meal. These data support the possibility that R-tofisopam may have a low propensity to slow basal, nonstimulated colonic propulsion in humans, and thus may have a lessened tendency to cause constipation than some medications currently used for the treatment of IBS.

### Example 4

### Evaluation of R-Tofisopam in Visceral Hypersensitivity, Pain, and Bloating in the Rat

The effects of (R)-tofisopam and (R,S)-tofisopam were evaluated for alleviating visceral hypersensitivity, pain, and bloating. In the visceral hypersensitivity, pain and bloating test, colonic distension is induced by inflating a balloon intra-colonically 2 hours and 30 minutes after intra-colonic application of 1% acetic acid (Langlois et al. Eur. J. Pharmacol. 324: 211-217 (1997); Langlois et al. Eur. J. Pharmacol. 318: 141-144 (1996)). The acetic acid makes the colon hypersensitive, modeling the visceral hypersensitivity seen in IBS. The colonic distension models the pain and bloating seen in IBS, and causes abdominal contractions in rats. In the control group 13.8±2.0 abdominal contractions were measured over the 10-minute period of observation. When rats are treated with U-50,488H (10 mg/kg), administered orally 60 minutes before colonic distension, the number of abdominal contractions was markedly reduced (2.5±0.8 versus 13.8±2.0 abdominal contractions in the control group, i.e. 82% reduction, p <0.01).

RS-tofisopam (32 mg/kg) did not significantly modify the number of abdominal contractions as compared with the vehicle control group. R-tofisopam (32 mg/kg p.o.) decreased the number of abdominal cramps as compared with control, significantly so at the dose tested (9.5±1.7 cramps, respectively, versus 18.8±−1.8 cramps in the vehicle control group, i.e. 49% decrease, p<0.001). In conclusion, R-tofisopam (32 mg/kg p.o.) exerted protective effects against visceral hypersensitivity, pain, and bloating induced by colonic distension in the rat.

### Example 5

### Evaluation of (R)-Tofisopam in Reducing Gastric Ulcer Formation

The (R)-enantiomer was evaluated for its ability to reduce stress-induced ulcer formation. In total, four studies were conducted. In each study, a typical benzodiazepine (clobazam) was used as a reference standard, and a control group received saline. The four studies were identical in design, differing only in compounds and/or the doses tested. A description of the basic study design is as follows.

After being deprived of food and water for approximately 24 hours, rats were put into restraint chambers positioned inside Plexiglass™ cylinders that were placed vertically in water at 22±1° C., with the rat immersed up to its neck. After remaining 1 hour in the water, rats were sacrificed by cervical dislocation and their stomachs were removed and scored for the presence of irritation or ulcers (the “ulcer score”), according to a five-point scale (0=no ulcers or irritation, 1=irritation, 2=1 or 2 ulcers, 3=3 or 4 ulcers, 4=more than 4 ulcers). The percentage of stomachs showing ulcers, and the ulceration index (ulcer score x percentage of stomachs showing ulcers) were also calculated. Test compounds were administered IP 30 minutes prior to water immersion.

In one set of experiments, R-tofisopam, as well as RS-tofisopam, were tested. Doses ranged from 8 to 128 mg/kg i.p. Clobazam (16 and 32 mg/kg) was used as reference compound, and saline solution was used as a control. Data from the first of these experiments are shown in Table 4.

TABLE 4
Effects of Clobazam and R-, and RS-Tofisopam in the Water
Immersion Stress-Induced Ulcers Test in the Rat (N = 8 Rats/Group)

| | Score for the | | |
|---|---|---|---|
| | Presence of Ulcers | % of Stomachs | Ulceration |
| Treatment | Mean ± SEM | Showing Ulcers | Index |
| Vehicle (saline) | 3.6 ± 0.3 | 100% | 363 |
| RS-tofisopam | | | |
| 8 mg/kg | 3.4 ± 0.3 | 100% | 338 |
| 16 mg/kg | 3.4 ± 0.3 | 100% | 338 |
| 32 mg/kg | 3.5 ± 0.3 | 100% | 350 |
| 64 mg/kg | 3.1 ± 0.3 | 100% | 313 |
| 128 mg/kg | 2.4 ± 0.4* | 75% | 178 |
| R-tofisopam | | | |
| 8 mg/kg | 3.6 ± 0.3 | 100% | 363 |
| 16 mg/kg | 3.1 ± 0.5 | 88% | 273 |
| 32 mg/kg | 3.1 ± 0.5 | 88% | 273 |
| 64 mg/kg | 1.7 ± 0.6* | 29% | 49 |
| 128 mg/kg | 1.4 ± 0.2*** | 38% | 52 |
| Clobazam | | | |
| 16 mg/kg | 3.6 ± 0.2 | 100% | 363 |
| 32 mg/kg | 2.6 ± 0.5 | 75% | 197 |

*p < 0.05;
**p < 0.01;
***p < 0.001.
Note:
statistics not performed on percent of stomachs showing ulcers or ulceration index.
Score:
0 = neither ulcer nor irritation;
1 = irritation;
2 = 1-2 ulcers;
3 = 3-4 ulcers;
4 = >4 ulcers
^{a}All rats died before water immersion.

As expected on the basis of the previous studies, a high ulcer score (3.6) was observed in the saline-treated control group following the 60-minute immersion period. Whereas pretreatment with R-tofisopam significantly reduced the ulcer score, R-tofisopam appeared to be more potent than RS-tofisopam in this test. Clobazam reduced the ulcer score slightly, but this effect was not statistically significant. A second experiment yielded similar results, confirming these findings.

This series of experiments clearly demonstrates significant activity for R-tofisopam in an animal model of stress/anxiety utilizing a gastrointestinal endpoint. These in vivo data lend further support for the potential utility of R-tofisopam for the treatment of gastrointestinal conditions in which stress may play a role, including IBS.

All references cited herein are incorporated by reference. The present invention may be embodied in other specific forms without departing from the spirit or essential attributes of the invention.

1. A method of treating or preventing altered bowel motility in an individual in need of such treatment, comprising administering to the individual an effective amount of enantiomerically-pure R-tofisopam, or a pharmaceutically-acceptable salt of such a compound.

2. The method of claim 1, wherein said altered bowel motility is associated with irritable bowel syndrome.

3. The method of claim 1, wherein said R-tofisopam is administered with a second drug.

4. A method of treating or preventing visceral hypersensitivity, pain, and bloating in an individual in need of such treatment, comprising administering to the individual an effective amount of enantiomerically-pure R-tofisopam; or a pharmaceutically-acceptable salt of such a compound.

5. The method of claim 4, wherein said visceral hypersensitivity is associated with irritable bowel syndrome.

6. The method of claim 4, wherein said R-tofisopam is administered with a second drug.

7. A method of treating or preventing gastrointestinal dysfunction related stress in an individual in need of such treatment, comprising administering to the individual an effective amount of enantiomerically-pure R-tofisopam; or a pharmaceutically-acceptable salt of such a compound.

8. The method of claim 7, wherein said R-tofisopam is administered with a second drug.

9. The method of claim 7, wherein said stress results in a gastric ulcer.

10. The method of claim 7, wherein said stress is associated with irritable bowel syndrome.

11. A method of treating or preventing irritable bowel syndrome, comprising administering to the individual an effective amount of enantiomerically-pure R-tofisopam; or a pharmaceutically-acceptable salt of such a compound.

12. The method of claim 11, wherein said R-tofisopam is administered with a second drug.
